(19) United States
(12) Patent Application Publication
(10) Pub. No.: US 2019/0224398 A1
(43) Pub. Date: Jul. 25, 2019

(54) Portable Dialysis System

(71) Applicant: Jalal Eddin Hakmei, Orangeburg, SC (US)

(72) Inventor: Jalal Eddin Hakmei, Orangeburg, SC (US)

(21) Appl. No.: 16/249,469

(22) Filed: Jan. 16, 2019

Related U.S. Application Data
(60) Provisional application No. 62/619,180, filed on Jan. 19, 2018.

(51) Int. Cl. A61M 1/16; A61M 1/26; A61M 1/36

(52) U.S. Cl. CPC A61M 1/1678; A61M 1/267; A61M 1/1629; A61M 2205/3337; A61M 2209/088; A61M 1/3638

(57) ABSTRACT

The present disclosure is a machine that permits intra-corporeal dialysis, which machine may be carried by the patient and include a canister of dialysate and a small pump. Filtration of toxins from blood takes place in the patient's own blood stream via a catheter. The catheter is inserted into a major blood vein. Dialysate is pumped from a first end of a canister into the central channel of the catheter. At the other end of the catheter, the dialysate passes to an annular region and reverses direction. The exterior wall of the exterior region of the catheter is permeable to blood, but not to the dialysate. Blood passes through the exterior wall of the catheter and is exposed to the dialysate in the annular region. There the dialysate absorbs toxins before it returns to the second side of the canister.

### TECHNOLOGICAL FIELD

This disclosure is related generally to dialysis and more particularly to dialysis machines.

### BACKGROUND

Dialysis machines are used to purify the blood of someone whose kidneys have failed. Blood purification is known as hemodialysis and the failure of kidneys to purify blood is called renal failure. A dialysis machine includes filters and a pump, plus tubes to connect to the patient. The blood is pumped from the patient, passed through the filters, which remove toxins, and then returned to the patient. This type of dialysis achieves the extracorporeal removal of waste products such as creatinine and urea and free water from the blood when the kidneys are in a state of kidney failure. The time a patient has access to a dialysis machine takes hours and must be rationed. Dialysis treatments are given periodically to such individuals and are essential for their continued existence. Delaying treatment may result in death. Moreover, dialysis machines are expensive and there are not enough of them for all patients to be treated on a proper schedule.

Dialysis membranes used clinically in the treatment of patients with renal failure account for by far the largest volume of artificial membranes used worldwide; more than 70 million square meters are used a year.

Almost all dialyzer filters now in use are of the hollow-fiber type. A hollow-fiber dialyzer contains a bundle of approximately 10000 hollow fibers, each with an inner diameter of about 200 microns when wet. The membrane thickness is about 20-45 microns, and the length is 160-250 mm. The walls of the hollow fibers function as the dialysis membrane. Various materials, including cellulose-based materials and synthetic polymers, are used for dialysis membranes.

A better way to perform dialysis would be an advantage for those with renal failure.

### SUMMARY

The present disclosure is a new dialysis machine. According to the present disclosure, the filtration is moved into the patient's own blood stream via a catheter that is inserted in a major blood vein. A dialysate is pumped from a canister carried by the patient into a central channel of the catheter. At the end of the catheter, it passes to an annular region running in the reverse direction. The exterior wall of the catheter is impermeable to cells in the blood stream but allows small and middle weight molecules to pass through into the dialysate transparent to blood but not to the dialysate. Blood passes through the wall of the catheter and is exposed to the dialysate. The dialysate absorbs toxins as it returns to the canister.

A feature of the disclosure is that dialysis takes place in a catheter in the patient's vein rather than in a machine external to the patient's body. Accordingly, the patient has more flexibility as to when, where and for how long that patient undergoes dialysis.

Another feature of the disclosure is a structured catheter that receives pumped dialysate and exposes it to blood flowing in a vein of a patient before returning the used dialysate to the exterior of the patient.

A feature of the disclosure is a dialysate canister that has a moving diaphragm that slides axially from one side of the diaphragm to the other as the fresh dialysate is pumped into the patient from one side of the canister and used dialysate is received back from the patient on the other side of canister.

Another feature of the disclosure is that the pump requirements are reduced from that of present dialysis machines because less blood is being processed. The pump is therefore smaller and may be easily carried. While complete dialysis may take longer, the patient may undergo dialysis at night while sleeping.

A feature of the disclosure is that the catheter brings the fresh dialysate into contact with the patient's blood in one channel and then exposes the dialysate to the patient's blood in a closed loop so the dialysate does not enter the blood.

These and other features and their advantages will be apparent to those skilled in the art of dialysis from a careful reading of the Detailed Description accompanied by the following drawings.

### BRIEF DESCRIPTION OF THE DRAWINGS

In the figures,

FIG. 1 is a schematic drawing of a prior art dialysis machine showing the connections between the components of the prior art dialysis machine and the patient;

FIG. 2 is a schematic drawing of the present dialysis system, according to an aspect of the disclosure;

FIG. 3 is a longitudinal cross sectional view of the present catheter, according to an aspect of the disclosure;

FIGS. 4A and 4B show a lateral and an end cross sectional view of the present catheter, according to an aspect of the disclosure;

FIG. 5 is a cross-sectional view of the canister, according to an aspect of the disclosure.

### DETAILED DESCRIPTION

The present disclosure describes a dialysis system wherein the dialysis, that is, the removal of the toxins from the patient's blood, takes place in the patent rather than external to the patient.

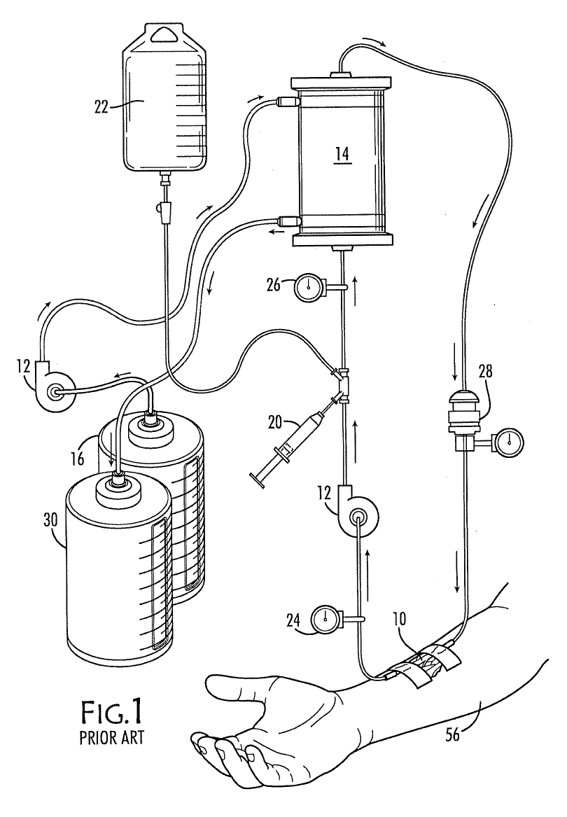

FIG. 1 shows in a schematic view the components of a prior art dialysis machine connected to a patient. According to the prior art system, blood is pumped by a pump 12 from a patient's blood vessel 10 to a dialyser 14 external to the patient. Dialyser 14 is a filter that removes toxins from the blood. Dialyzer 14 is connected to a supply 16 of dialysate, which is a liquid that toxins attach to. In order to prevent clotting of the blood when it is in dialyzer 12 and outside the body, an anti-clotting agent 20, such as heparin, is added the blood before as enters the dialyser 14. A small quantity of saline 22 may also be added to the blood of the patient as it enters dialyser 14.

The dialysate is pumped into dialyser 14 and flows counter to the direction of the blood flow as it interacts with the blood and absorbs toxins as it passes through and exits the dialyzer. The used dialysate then flows into a used dialysate container 18. Pressure gauges 24, 26, continuously monitor and regulate the system. An air trap 28 prevents air from entering the body.

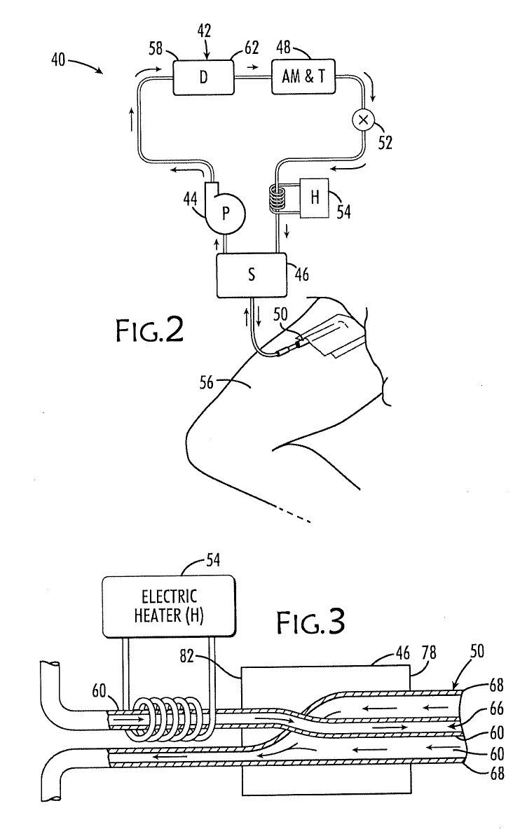

FIG. 2 is a schematic diagram of the present dialysis system, generally indicated by reference number 40. System 40 include a canister (D) 42 of dialysate, a pump (P) 44, a separator (S) 46, an entrained air trap (AM&T) 48 and a catheter 50. Catherter 30 is shown in FIG. 2 inserted in a larger blood vessel of the patient's body 34 such as a femoral vein or jugular vein. System 10 may include a heating element (H) 54 to warm the dialysate before it is infused into catheter 50.

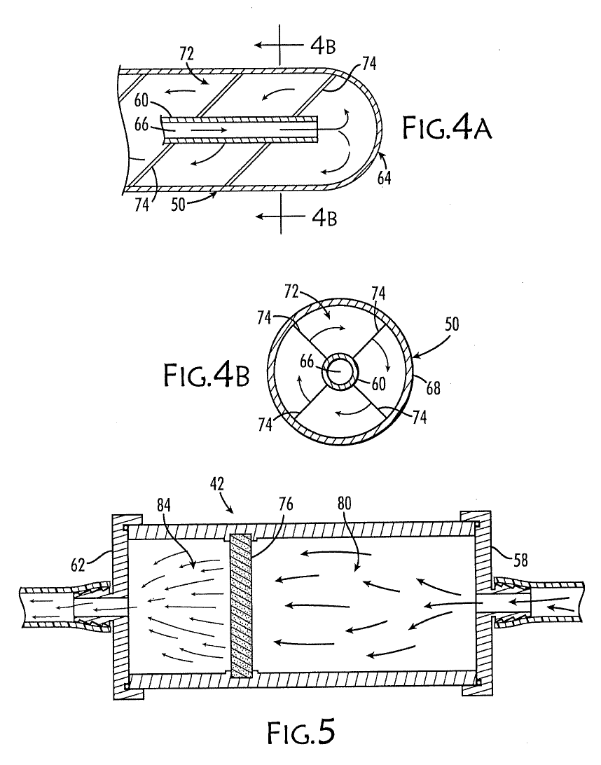

In FIG. 2 blood flows around and through part of catheter 50 but otherwise remains in the patient's body 56. Dialysate flows through catheter 50 in two directions, best seen in FIGS. 3, 4A, and 4B. Dialysate flows from a first end 58 of canister 42 through pump 44 and then into separator 46, the function of which will be described presently. A valve 52 in line with the pump controls the amount of pressure in the line because the pump generates a negative pressure in drawing blood from the body of the patient.

In separator 46, as best seen in FIG. 3, 4A, and 4B, dialysate 60 will enter catheter 50. After leaving separator 46 where it absorbed toxins from the blood, the used dialysate passes through air trap 48 and enters second end 62 of canister 42.

The “fresh” dialysate, that is, dialysate that has not passed through catheter 50, is directed by separator 46 into the central channel 66 of catheter 50 where it flows to the tip 64 of catheter 50 (FIGS. 4A and 4B) and then turns radially outward and flows in the reverse direction flowing through an annular region until it leaves catheter 50, flowing toward second end 62 of separator 46.

The outer wall 68 of catheter 50 is permeable to a cellular components of blood. The dialysate can pass through but due to negative pressure most of it will not. The inner wall 60 of catheter 50 is not permeable to blood or dialysate. In annular region 72, there may be baffles and passages (not shown) that create greater interaction between the blood and the dialysate so as to facilitate absorption of toxins. Annular region 72 of catheter 30 may hold nepheline filter material. The cross-sectional area of annular region 72 may be larger than that of central channel 66 to allow additional time for blood and dialysate to interact. Blood may flow in a serpentine or circular path as directed by internal structure 74. A reasonable amount of experimentation may be required to establish a suitable residency time for the dialysate and blood to interact that allows the dialysate to absorb a useful amount of toxins before it is returned to canister 42.

The apparatus includes pump 44, perhaps one that would be small enough and light enough to fit into a backpack. Canister 42, as shown in the cross sectional view of FIG. 5, has a slidable diaphragm 76 that adjusts to the decrease in volume 80 of fresh dialysate on first side 82 is replaced by the in-flux of volume 84 of used dialysate on the second side 86. System 40 may also include a belt and a vest (not shown) to hold pump 44 and canister 42 in place during use.

Treatment may take longer with the present system 10, but the patient may be able to connect himself or herself to it, the apparatus may be less costly, and thus patients may have greater access to the device.

What is claimed is:

1. A dialysis system, comprising:
(a) a canister, said canister having a first end and an opposing second end, said first end and said second end being separated by a slidable diaphragm;
(b) a pump connected to said first end of said canister;
(c) a catheter having a proximal end and an opposing distal end, said catheter having a central channel and an annular channel communicating with said central end, said catheter having an outer wall permeable to blood and said central channel having a wall impermeable to blood.

2. The dialysis system of claim 1, further comprising dialysate in said canister.

3. The dialysis system of claim 1, further comprising an air monitor.

4. The dialysis system of claim 1, further comprising a separator connected to said catheter and said pump and said second side of said canister, said separator separating flow of used dialysate from said fresh dialysate.
